(19) United States
(12) Patent Application Publication
(10) Pub. No.: US 2012/0253391 A1
(43) Pub. Date: Oct. 4, 2012

(54) Instrument Kit For Knotless Fixing Of Tissue To A Bone

(76) Inventor: Sebastian Frey, Waghaeusel (DE)

(21) Appl. No.: 13/439,445

(22) Filed: Apr. 4, 2012

(30) Foreign Application Priority Data
Apr. 4, 2011 (DE) 10 2011 016 659.9

(51) Int. Cl. A61B 17/04

(52) U.S. Cl. 606/232

(57) ABSTRACT

An instrument kit serves for knotless fitting of a tissue to a bone. A pin-shaped anchor element has a suture connected to it. The suture has a loop portion formed into an annular body. An internal diameter of said annular body is chosen in that the pin-shaped anchor element can be moved along its longitudinal axis through said annular body. A length of a section of the suture between the anchor element and the annular body is chosen in that after said anchor element has moved through said annular body, a resulting new loop lies circumferentially and tightly around a strand-shaped tissue section to be fixed at said bone.

### BACKGROUND OF THE INVENTION

The invention relates to an instrument kit for knotless fixing of tissue to a bone, with a pin-shaped anchor element, which has a longitudinal axis and which is connected to a suture having a loop portion at the end thereof.

With instrument kits or anchor systems of this kind, a tissue that has become detached from a bone can be fixed to the latter again.

This need arises in injuries to the shoulder, the hip or the like. There, cuff-like or collar-like tissue regions are present around the joints and are fused to the bone. When injuries occur, principally sports injuries, strand-shaped portions of the tissue are usually detached from the bone on one side.

A typical injury to the shoulder joint occurs, for example, in handball players who are tackled when throwing a shot for goal.

The basic principle of fixing involves providing an anchor which is connected to a suture. The suture serves to connect the anchor to the detached tendon endpiece.

The anchor element itself is introduced into the bone, either being fitted into a previously drilled bore or being driven with impact directly into the bone. In this way, the detached tendon, which is connected to the anchor via the suture, is fixed to the bone again.

In the early days of this technique, the anchor, with loose suture ends protruding from it, was driven into the bone, and the suture ends were connected to the detached tissue section and then knotted.

It was found that these knots form undesired elevations under the skin and disrupt the subsequent healing process.

Therefore, this technique was refined in the sense that so-called knotless anchor systems were developed. In these, a suture in the form of a loop protrudes from the anchor, and the loop is connected to the tendon section that is to be fixed.

The torn-off tissue is therefore tensioned, or fixed as tightly as possible on the bone, not by means of a suture being suitably tensioned and then knotted, but instead by virtue of the fact that the loop can be tensioned by suitable configuration of the anchor element.

Devices of this kind or instrument kits of this kind are known from DE 10 2006 010 116 A, EP 1 199 036 B1 or WO 2005/102190 A2, for example.

### SUMMARY OF THE INVENTION

It is an object of the invention to refine an instrument kit of this kind for knotless fixing in such a way that the fixing procedure can be performed safely, easily and in a targeted manner.

This object is achieved by an instrument kit for knotless fixing comprising a pin-shaped anchor element which can be anchored in a bone, said pin-shaped anchor element has a longitudinal axis extending along said pin-shaped anchor element and an outer diameter; a suture connected to said pin-shaped anchor element, said suture having a loop portion, said loop portion being formed into an annular body, said annular body having an internal diameter being chosen in that said pin-shaped anchor element can be moved along its longitudinal axis through said annular body; a length of a section of said suture between said anchor element and said annular body is chosen in that, after said anchor element has moved through said annular body, a resulting new loop lies circumferentially and tightly around a strand-shaped tissue section to be fixed at said bone.

These measures have numerous advantages.

The basic structure is such that the anchor element is connected, via a portion of the suture having a defined length, to the loop portion which is formed into an annular body. By choosing the internal diameter of the annular body in such a way that the anchor element can be pushed through this annular body, a new loop is created after the anchor element has been pushed through.

This now newly formed loop can be used to be placed around a strandshaped tissue section. By pulling on the anchor element already moved through the annular body, this loop becomes ever tighter, i.e. arranges itself ever tighter around the strand-shaped tissue section that is to be fixed. This is controlled in practice by the depth to which the anchor element is driven into the bone. Since the operating surgeon knows approximately how thick this strand-shaped tissue section is, he also knows what length to choose for the portion of the suture between the annular body and the anchor element in order to form a loop that tightly encloses the strand-shaped tissue section and accordingly draws the latter with tensioning to the annular body or in the direction of the anchor element.

It is possible for this length to be individually adjusted in situ or to be set already at the place of manufacture on the basis of experience. It is known that, for example, the height of a cuff surrounding a human shoulder has a certain measurement, i.e. it is also then known what diameter a strand of tissue has that is partially torn off from this cuff.

During handling, the assembly composed of the anchor element, of the suture portion protruding therefrom and of the annular body, is placed laterally around the strand-shaped tissue section. The annular body can already be placed on the bone surface at the site where the anchor element is intended to be driven in. Thereafter, the anchor element is placed centrally in the annular body, as a result of which the portion of the suture between anchor element and annular body has already arranged itself around the strand-shaped tissue section. By driving the anchor element through the annular body and into the bone, this new loop is then drawn tight around the strand-shaped tissue section and draws the latter onto the bone. The depth to which the anchor element is driven into the bone thus determines the degree of tensioning of the new loop for fixing the strand-shaped tissue section.

The operating surgeon does not have to perform any complicated assembly steps, as is the case, for example, in anchor pieces composed of multi-part elements. The fixing procedure is easy to carry out, i.e. the assembly composed of anchor element, suture and annular body simply has to be placed around the strand-shaped tissue section, and the anchor element then driven through the annular body into the bone.

The annular body, formed from the suture itself, can, with suitable material of the suture, be preformed as an annular body if the suture material has a correspondingly sufficient dimensional stability. This annular body can also be configured by the fact that the portion between the anchor element and the annular body to be configured is formed by two suture portions of a single suture, which as a whole is configured as a loop. If, as has been mentioned, the material allows the annular body to be configured with suitable dimensional stability, the length portion of the suture between the annular body and the anchor element is then configured as a double strand. Suitable auxiliary measures can also be taken to maintain the annular body as such; for example, at the site of the annular body from which this double strand leads away, a small clip or a fastening point is formed, for example by a spot of adhesive or the like.

Bearing in mind that, after the anchor element has been forced through the annular body, the latter opens and this suture portion forms part of the loop which as a whole is placed around the strand-shaped tissue section, this length portion accordingly has to be added on, since otherwise the anchor element would have to be driven unnecessarily deeply into the bone.

It is important that this annular body is present at the outset in order to provide the operator with a clear orientation as to where the anchor element should be guided through. In the case where the ring geometry is thereafter lost, the half length of the circumference of this annular body must then be added to the length of the portion of the suture between the original annular body and the anchor element, in order finally to obtain a loop length that is exactly sufficient to be placed circumferentially and tightly around the strand-shaped tissue section and to build up the sufficient tension by suitable driving-in of the anchor element in the bone. The length of the suture, i.e. the length of the suture portion plus the half circumference of the annular body, is to be chosen such that the tissue section is securely enclosed.

In another embodiment of the invention, the loop portion is guided around a ring element.

This measure has the advantage that the loop geometry is determined by this additional ring element, such that, even with suture materials that are very slack, the ring geometry is still maintained. This ring element also remains in the body after the anchor element has been driven through the ring element.

This ring element is an element that is easy to handle, i.e. the operating surgeon can grip it by hand, for example, after he has placed the assembly around the strand-shaped tissue section, and can place it at a very defined location on the bone, which location is favourable for the driving-in of the anchor piece.

In another embodiment of the invention, the ring element is designed as a ring on whose circumferential outer face a recess is present, into which the loop portion can be placed.

This measure has the advantage that the original loop portion can already be placed into the ring element or into the outer recess at the place of manufacture, and the corresponding annular body is then formed from the suture material.

In another embodiment of the invention, the recess is designed in such a way that the suture portion is secure against slipping off.

This measure has the advantage that the suture, for example at the place of manufacture, is pressed with a certain pressure into the recess, such that this assembly is then maintained firmly during handling, and the ring does not come loose from the suture, or vice versa.

This handling is first of all the packaging procedure after production, the opening of the package at the place where the operation is being performed, the handing to the operating surgeon, and then also the handling by the operating surgeon upon application to the tissue section. In all these manoeuvres, the suture portion remains firmly secured in the ring.

This considerably facilitates the handling of the device.

In another embodiment of the invention, the anchor element has a proximal attachment site to which the suture can be attached.

This measure has the advantage that the length of the suture portion, in particular the length of the portion between the anchor element and the annular body, can be adapted using this attachment site. Thus, with one and the same physical embodiment of anchor element and ring, an assembly can be made available which is suitable, for example, for placing around a relatively thick strand of tissue or which, with a suitably shorter design of this portion between anchor element and annular body, is suitable for placing around a substantially thinner strand.

This also opens up the possibility that the thickness of the tissue section or strand to be secured can first of all be determined directly in situ, and then the suture can be applied to the proximal attachment site so as to provide optimal conditions.

Optimal conditions means that the loop formed after the anchor element has been pushed through the ring element bears tightly around the outer face of the tissue to be fixed, such that this tissue is held securely and, at the same time, the tensioning is already built up when the anchor is driven to the full extent into the bone. This means that the proximal end of the anchor comes to lie at the height of the bone surface. This then results in the anchor element being introduced into the bone in a manner that is as atraumatic as possible, and without its having to penetrate unnecessarily deeply into the bone. This leads not only to much less traumatic anchoring, but also to a very high degree of flexibility in respect of the tissue sections that are to be fixed.

In another embodiment of the invention, the attachment site is a circumferential groove on the outer face of the anchor body, into which groove the anchor-side end of the suture can be placed.

This measure has the advantage of providing a relatively stable form-fit connection between the suture and the anchor element.

The end of the suture directed towards the anchor element is wound once round this circumferential groove and then suitably secured. This securing can easily be provided by a knot or some other clamping piece, which can already be made available at the place of manufacture. In the case where a knot is to be made, the anchor element then has a corresponding recess in order to be able to receive this knot, such that the latter does not protrude laterally. However, the principle of the knotless anchor is then still maintained, since this is understood as meaning that no new knot has to be provided during the actual fixing of the tissue. This is the case here, since the fixing is provided by the newly created loop after the anchor element has been driven through the annular body.

In another embodiment of the invention, the proximal end of the anchor element has an engagement feature for a driving-in tool.

This measure has the advantage that a further component of the instrument kit, namely a driving-in tool, can be applied to the anchor element in order to drive the latter through the annular body into the bone.

In another embodiment, the anchor element is designed as a solid cylindrical body, of which the distal end is designed as a conical tip.

This measure has the advantage, for example, that the anchor element can be driven directly into the bone via the tip, without a bore first having to be prepared.

This also increases the flexibility, since the operating surgeon, after placing the assembly on the tissue section that is to be fixed, can first of all look for a suitable location on the bone where he places the annular body and then accordingly applies the tip of the anchor element in order to drive the latter in. If a bore had to be prepared beforehand, he would then be restricted to the position and orientation of this bore. This therefore also increases the flexibility of use of such an instrument kit.

In another embodiment of the invention, the engagement feature is designed as a bore extending through the body of the anchor element in the longitudinal direction.

This measure, known per se, has the advantage that the engagement between the driving-in tool and the anchor element can take place quite simply via this bore, namely by pushing a corresponding projection of the driving-in tool into this bore.

In another embodiment of the invention, the bore extends completely through the body, and the conical tip is designed as a truncated cone, and a driving-in tool inserted into the bore has a tip, which supplements the truncated cone so as to form a pointed cone.

This measure has the advantage that the tip of the driving-in tool protruding past the truncated cone can be suitably stable and solid or made from a metal, so as to be able to drive the assembly easily and in a targeted manner into the bone.

After the driving-in tool has been removed, the anchor element remains in the body, with only the blunt truncated cone then representing the distal front end of the anchor element. In this way, the anchor element itself can be made from other materials, including materials that are less impact-resistant. This also makes it possible in general to design the anchor element as a relatively small, weak component, such that fixing procedures of this kind can also be performed, for example, on bone areas that do not have such a large surface area as the shoulder.

In another embodiment of the invention, a holding device is provided, to which a driving-in tool connected to the anchor element is connected, and which at the distal end has a clamp into which the ring element and the suture portion can be laterally clipped.

This measure has the advantage that the holding device holds the driving-in tool in a defined orientation, in which the anchor element can then be applied to the driving-in tool. By means of the clamp at the distal end, the ring element can at the same time be brought into the correct orientation, such that anchor element and ring element are in an orientation to each other in which the anchor element simply has to be driven through the ring element by means of a linear advance motion. By forming a clamp in which the ring element can be clipped, this assembly is easy to produce and, of course, then also easy to dismantle after the anchor element has already been driven either partially or completely through the ring element.

In another embodiment of the invention, the holding device has a guide, extending laterally with respect to the longitudinal axis, for the suture portion between annular body and anchor element.

This measure has the advantage that, by means of this guide, this suture portion between annular body and anchor element lies in a defined orientation, which then makes it easier for the operating surgeon to move this suture portion laterally towards the tissue that is to be fixed and apply it thereto.

In another embodiment of the invention, the guide is designed as a vertical bracket which bulges out laterally and whose side facing the suture portion has a recess for receiving the suture portion.

This measure has the advantage that this suture portion can be held and guided in the recess of the outwardly bulging bracket.

This means, for example, that this complete assembly of the instrument kit can already be produced before the operation. If, before the surgical intervention, it is to be individually adapted to the diameter of the tissue, this can be done by an assistant. The prepared instrument kit is then handed to the operating surgeon, who then guides it laterally onto the tissue strand that is to be fixed, places the clamp with the ring element onto the bone, and then simply has to impact the driving-in tool in order to drive the latter, together with the anchor element to be secured thereon, in a targeted manner through the ring element into the bone.

In another embodiment of the invention, the driving-in tool is mounted in a vertically adjustable manner on the proximal end of the bracket.

This measure has the advantage of permitting further individual adaptability.

In another embodiment of the invention, the clamp, which is arranged at the proximal end of the bracket, and the annular body of the suture portion are held in such an orientation that the annular body can be placed flat on an outer face of the bone.

This measure has the advantage that the instrument kit can be guided towards, oriented with respect to and placed on the bone surface much more easily by the operating surgeon.

In another embodiment of the invention, the annular body is held in the clamp in an orientation in which the longitudinal axis of the anchor element is centrally perpendicular with respect to the plane of the ring.

This geometry allows the operating surgeon to apply the clamp in a targeted manner and also provides him with a visual indication of the impaction direction or drive direction in which the anchor element is to be driven in. If he knows approximately how long this anchor element is, he then knows the orientation in which he has to apply the instrument kit in order in each case to have sufficient bone substance available into which the anchor element can be driven.

This also considerably increases the flexibility of use of the instrument kit.

It will be appreciated that the aforementioned features and the features still to be explained below can be used not only in the respectively cited combination but also in other combinations or singly, without departing from the scope of the present invention.

### BRIEF DESCRIPTION OF THE DRAWINGS

Illustrative embodiments of the invention are explained in more detail in the description below and are depicted in the drawing, in which:

FIG. 1 shows a perspective view of the assembly composed of an anchor element and of a suture portion protruding laterally in an arc shape therefrom and opening into the annular body,

FIG. 2 shows a corresponding view where, in order to stabilize the annular body, the latter is placed around a ring element,

FIG. 3 shows the complete assembly of an instrument kit, an embodiment being depicted here in which the suture is secured on the anchor element in a circumferential groove, is guided as a double strand to the annular body, and this double strand is received in a lateral guide piece of a bracket, and at the same time a driving-in tool is mounted on the anchor element,

FIG. 4 shows a very schematic side view of the structural elements from FIG. 1, this view illustrating how the portion of the suture between anchor element and ring element can be placed around a strand-shaped tissue that is to be fixed, and

FIG. 5 shows the anchor element with tissue fixed to the bone.

### DETAILED DESCRIPTION OF PREFERRED EMBODIMENTS

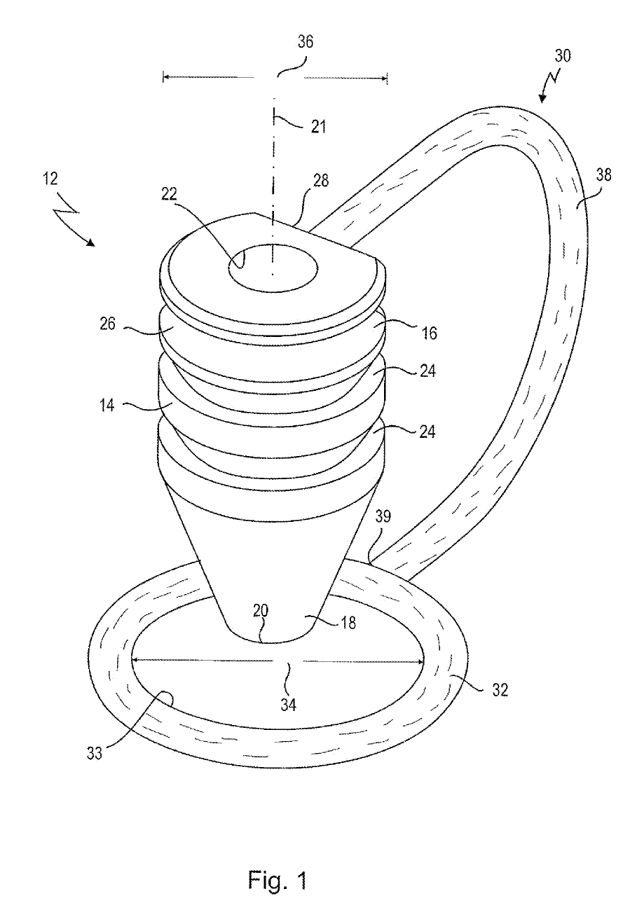

An instrument kit shown in FIGS. 1 to 5 is designated overall by reference number 10.

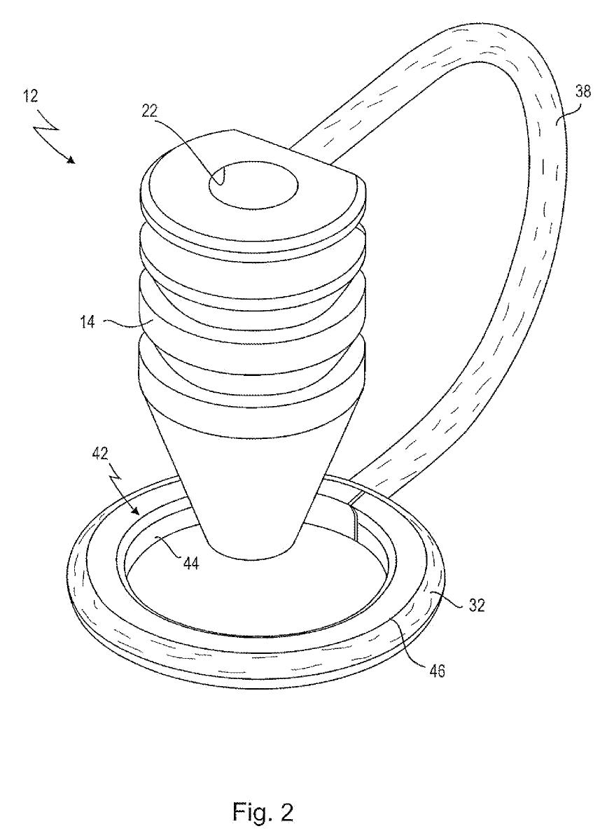

An important structural group of the instrument kit 10 is the anchor element 12 shown in FIGS. 1 and 2 and in the form of a pin 14.

The pin 14 has a solid cylindrical body 16, which merges at the distal end into a narrowing tip 18. The tip is designed as a truncated cone 20.

A continuous bore 22 extends centrally all the way through the anchor element 12 along the longitudinal axis 21 of the latter. This bore 22 can also be provided with an internal thread at the proximal end area.

On the outer face of the cylindrical body 16, various indentations 24 are provided, which are intended to act as barbs after the anchor element 12 has been driven into a bone.

Near its proximal end, the cylindrical body 16 has a circumferential annular groove 26. In this proximal end area, the cylindrical body 16 has a flattened face 28.

The anchor element 12 is connected to a suture 30. At its end remote from the anchor element 12, this suture 30 has a loop portion 32, which is formed into an annular body 33.

The internal diameter 34 of the annular body 33 is chosen such that it is slightly greater than the external diameter 36 of the cylindrical body 16, with the result that the cylindrical body 16, directed downwards in the view in FIG. 1, can be easily moved through the interior of the annular body 33.

A laterally outwardly curved portion 38 of the suture extends between the cylindrical body 16 and the annular body 33.

FIG. 1 shows that the annular body 33, as a closed ring, consists of the suture material, which is connected fixedly to the portion 38.

At its end remote from the annular body 33, the portion 38 is secured on the proximal end of the cylindrical body 16.

This can be done, for example, by means of this end being inserted into an opening (not shown) in the cylindrical body 16 and secured there, which can be carried out already at the place of manufacture.

However, it is also possible to secure this end of the suture 30 on the anchor element 12 by placing it in the annular groove 26 and knotting it in the area of the flattened face 28. In this embodiment, the length of the portion 38 is then correspondingly variable.

However, it is also possible to design the suture 30 such that, starting from the cylindrical body 16, it is designed as a double strand, which then divides to form the annular body 33. However, measures then have to be taken to maintain the annular body 33 as a ring. This can be achieved, for example, by a corresponding plastic deformability of the material of the suture. At the branch site 39, i.e. at the site where the double strand leaves the annular body, a corresponding clip or a spot of adhesive can be applied in order to maintain the ring geometry of the annular body 33. In this case, the annular body is then designed as one strand.

FIG. 2 shows an embodiment in which the annular body 33 is placed around a ring element 42.

The ring element 42 consists here of a metallic, stiff and stable ring 44, on the circumferential outer face of which a recess 46 is provided, for example a groove, into which the annular body 33 can be placed or pressed in with a snug fit from the outside.

Depending on the requirements at the place of manufacture, the annular body 33 can be designed as a continuous prefabricated ring, or the ring 44 can be designed as a slotted ring, so as to make it easier for the suture to be pushed over or inserted. If the material of the suture 30 has a certain elasticity, this annular body 33 can be simply pushed over a closed ring. The configuration in FIG. 2 ensures that the annular body 33 maintains its ring geometry even when the suture material is flexurally slack, since this ring geometry is predefined by the ring element 42.

It is also possible for the assembly shown in FIG. 2 to be already prefabricated as such at the place of manufacture.

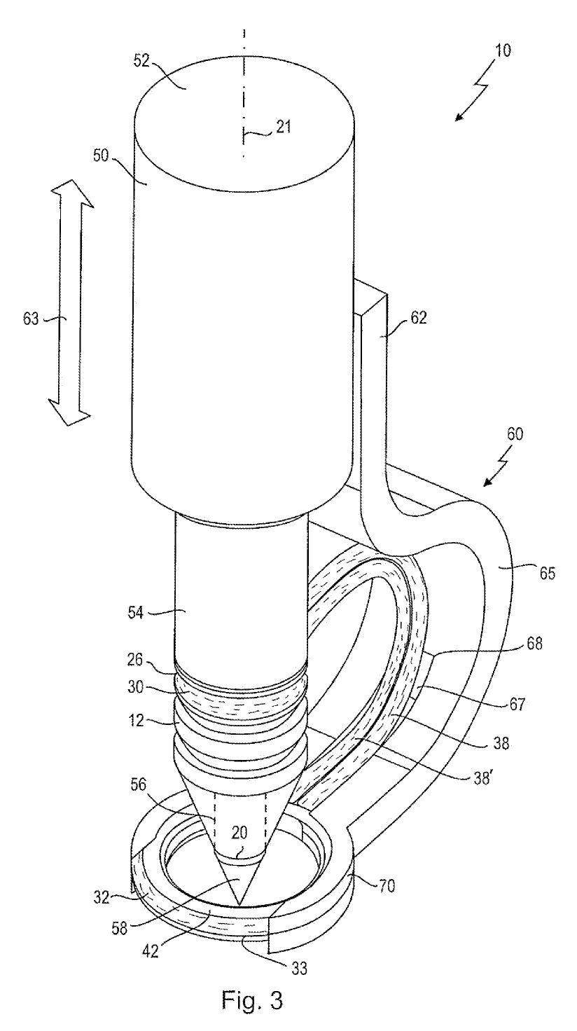

FIG. 3 now shows how further structural parts of the instrument kit 10 are designed and handled.

Here, a driving-in tool 50 can first be seen which, at the proximal end, has a strike head 52, which merges at the distal end into a slightly thinner bar 54.

From the distal end of the bar 54 itself, a rod 56 projects which is provided at its outer end with a tip 58.

The external diameter and length of the rod 56 are dimensioned such that it can be pushed through the continuous bore 22 from proximal to distal. If the aforementioned internal thread is present in the bore 22, a corresponding external thread is present on the outer face of the rod 56, such that the screw connection provides a firm and secure fit of the anchor element 12 on the driving-in tool 50. After the driving in, the driving-in tool 50 is unscrewed from the anchor element 12 that has been driven into the bone. The tip 58 protrudes past the truncated cone 20 of the anchor element 12 and is designed such that it supplements the truncated cone 20 so as to form a pointed cone. The material of the rod 56 and of the tip 58 is produced from hard-wearing metallic material, for example medical-grade steel.

The annular shoulder (not indicated here) in the transition from the bar 54 to the smaller-diameter rod 56 serves as an abutment on the flat proximal end of the cylindrical body 16 of the anchor element 12.

The driving-in tool 50 is mounted on a lateral holding device 60 in a vertically adjustable manner, as is indicated by the double arrow 63.

For this purpose, the holding device 60 has a rail 62, on which the strike head 52 is guided in a vertically adjustable manner.

The rail 62 continues in the form of a laterally outwardly bulging bracket 65, at the lower end of which a clamp 70 is arranged. The size, the opening and the orientation of the clamp 70 are such that the assembly of ring 44 and annular body 33 of suture material shown in FIG. 2 can be clipped laterally therein.

FIG. 3 shows that the portion of the suture 30 between the annular body 33 and the anchor element 12 is designed as a double strand 38, 38′, which is guided once round the annular groove 26 and knotted on sides of the flattened face.

Here, the suture 30 is to be designed as an originally single-strand suture of a defined length, and it can then be placed from the outside into the ring element 42, and the strands 38, 38′ protruding therefrom are then guided in an inner recess 67 of the bracket 65 to the proximal end of the anchor element 12 and are secured there in the manner previously described.

In the state shown in FIG. 3, the instrument kit 10 is ready for use in a surgical intervention.

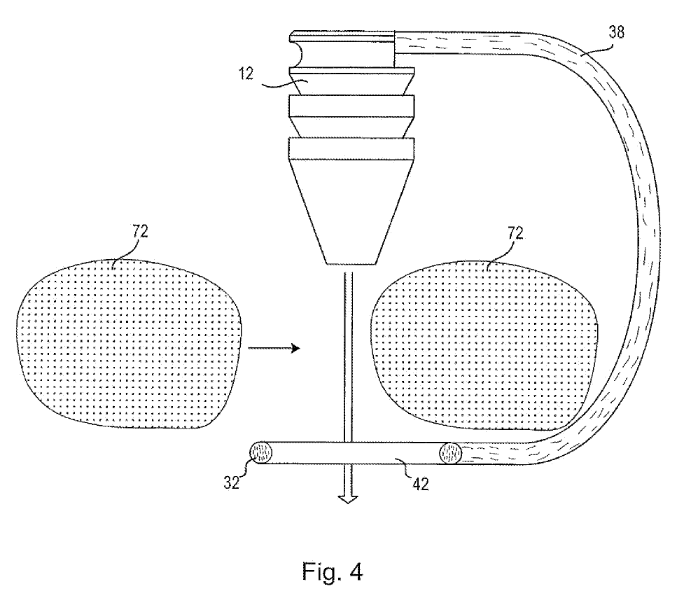

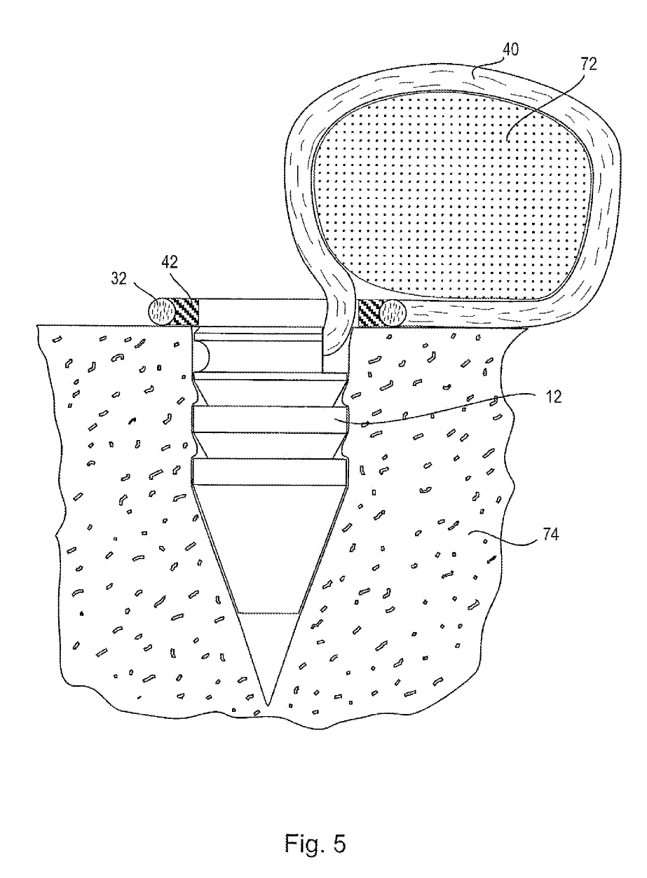

This is shown in a very schematic form in FIGS. 4 and 5 and, for the sake of clarity, without the actual driving-in tool and the holder.

By virtue of the vertical adjustability, the driving-in tool 50 and the anchor element 12 mounted thereon can be raised to such an extent that a tendon strand 72 that is to be secured is placed in the curved area or portion 38 of the suture 30. The instrument kit 10 can then be placed from one side onto the tendon strand 72, that is to say, in the view in FIG. 4, moved from the right to the left.

The end effect is that the tendon strand 72 is brought into this inner space. In the state of assembly shown in FIG. 3, the anchor element 12 can now be driven from above through the ring element 42. For this purpose, the underside of the ring element 42 is placed onto a bone surface, specifically at a site which is suitable for fixing the tendon strand 72 and also suitable for the driving-in of the anchor element 12. After it has been driven in, as can be seen in FIG. 5, the whole anchor element 12 has penetrated the bone 74.

The portion 38 has now fitted around the outer face of the tendon strand 72 and thus forms a loop 40 by which the tendon strand 72 is placed onto the bone 74 and thereby fixed.

It is clear from FIG. 5 that the ring element 42 remains on the outer face of the bone 74.

The necessary tensioning on the loop 40 can be achieved if the anchor element 12, from the position shown in FIG. 5, is driven slightly farther into the bone 74.

It is clear here that the circumferential length of the loop 40 corresponds approximately to the length of the portion 38, as is shown in FIG. 4. It is thus possible to accordingly select the length before the intervention, if the outer circumference of the tendon strand 72 is known.

If the aforementioned embodiment is used in which the suture is designed as a double strand in the area of the connection between the annular body 33 and the anchor element 12, and if no such ring element 42 is present, this suture area, i.e. along the length of the circumference of the annular body 33, can also be used to form the loop 40, which is then taken into consideration in the corresponding length of the portion 38. That is to say, if the annular body 33 can be made sufficiently stable, it is possible to use this instrument kit even without the ring element 42.

It is also possible for the suture area received in the ring element 42 to be used for securing, by means of this suture area being removed from the ring element 42. Then, half the length of the circumference of the ring element is to be added for forming the loop 40.

What is claimed is:

1. Instrument kit for knotless fixing of a tissue to a bone, comprising
a pin-shaped anchor element which can be anchored in a bone, said pin-shaped anchor element has a longitudinal axis extending along said pin-shaped anchor element and an outer diameter;
a suture connected to said pin-shaped anchor element, said suture having a loop portion, said loop portion being formed into an annular body, said annular body having an internal diameter being chosen in that said pin-shaped anchor element can be moved along its longitudinal axis through said annular body;
a length of a section of said suture between said anchor element and said annular body is chosen in that, after said anchor element has moved through said annular body, a resulting new loop lies circumferentially and tightly around a strand-shaped tissue section to be fixed at said bone.

2. Instrument kit of claim 1, wherein said internal diameter of said annular body being slightly greater than said outer diameter of said pin-shaped anchor element.

3. Instrument kit of claim 1, wherein said loop portion is guided around a ring element.

4. Instrument kit of claim 3, wherein said ring element is designed as a ring having a recess on its outer circumferential face, into which recess said loop portion can be placed.

5. Instrument kit of claim 4, wherein said recess is designed in such a way that said suture portion is secured against slipping off said recess when placed therein.

6. Instrument kit of claim 1, wherein said anchor element has a proximal attachment site to which said suture is attached.

7. Instrument kit of claim 6, wherein said attachment site has a circumferential groove at an outer face of a body of said anchor element into which circumferential groove said suture can be placed.

8. Instrument kit of claim 1, wherein a proximal end of said anchor element has an engagement feature for engaging a driving tool.

9. Instrument kit of claim 1, wherein a body of said anchor element is designed as a cylindrical body, having a distal end designed as a conical tip.

10. Instrument kit of claim 8, wherein said engagement feature is designed as a bore extending through a body of said anchor element in a direction of said longitudinal axis.

11. Instrument kit of claim 10, wherein said bore is designed as a bore extending completely through said body, and wherein said body has a conical tip designed as a truncated cone, and a driving tool inserted into said bore as a tip which supplements said truncated cone so as to form a pointed cone.

12. Instrument kit of claim 1, wherein a holding device is provided and, a driving tool connected to said anchor element can be connected to said holding device, and wherein said holding device has, at a distal end a clamp into which a ring element guiding said annular body of said suture can be laterally clipped.

13. Instrument kit of claim 12, wherein said holding device has a guide, extending laterally with respect to said longitudinal axis of said anchor element, said guide guides said section of said suture between said annular body and said anchor element.

14. Instrument kit of claim 13, wherein said guide is designed as a vertical bracket which bulges out laterally and a side of which facing said suture portion has a recess for receiving said suture section.

15. Instrument kit of claim 14, wherein said driving in tool being mounted in a vertically adjustable manner on a proximal end of said bracket.

16. Instrument kit of claim 15, wherein a clamp is arranged at a distal end of said bracket, said clamp holds said annular body of said loop portion in such an orientation that said annular body can be placed flat on an outer surface of a bone.

17. Instrument kit of claim 16, wherein said annular body is held in said clamp in an orientation in which said longitudinal axis of said anchor element is centrally perpendicular with respect to a plane of said annular body.
